(19) United States
(12) Patent Application Publication
(10) Pub. No.: US 2002/0039566 A1
(43) Pub. Date: Apr. 4, 2002

(54) Gel air freshener and method of manufacturing same

(76) Inventors: Carl Triplett, Scottsdale, AZ (US); Mengtao Pete He, Scottsdale, AZ (US); Debra Park, Mesa, AZ (US)

Correspondence Address: SNELL & WILMER, ONE ARIZONA CENTER, 400 EAST VAN BUREN ST., PHOENIX, AZ, 85004, US

(21) Appl. No.: 09/859,820

(22) Filed: May 16, 2001

Related U.S. Application Data
(60) Provisional application No. 60/204,585, filed on May 16, 2000.

(51) Int. Cl. A61L 9/00

(52) U.S. Cl. 424/76.1

(57) ABSTRACT

Improved gel compositions for delivering volatile materials such as fragrance oils, fragrance solutions, deodorants, insecticides, and the like into the surrounding air are disclosed. In general, such gel compositions do not include the use of cross-linking and/or chelating agents. In accordance with various embodiments of the present invention, hardness and resistance to syneresis of a translucent gel composition is significantly improved by employing a modified polysaccharide as the gelling agent. In accordance with other embodiments, also disclosed is the use of an amine oxide to reduce the total surfactant level of the gel. Preferred gel compositions, in accordance with various embodiments of the present invention, include a nonionic-based silicone antifoam agent to effectively control the production of foam during manufacture of the gel composition.

This application claims priority to U.S. Provisional Patent Application, Ser. No. 60/204,585 entitled “GEL AIR FRESHENER COMPOSITION AND METHOD FOR MANUFACTURING SAME” filed on May 16, 2000, which is incorporated herein by reference.

### FIELD OF THE INVENTION

The present invention generally relates to gel compositions. More particularly, the present invention relates to gel compositions useful as air fresheners having improved properties.

### BACKGROUND OF THE INVENTION

Aqueous-based, free-standing gels are one form of air freshener used to deliver fragrance into the air for achieving a desirably scented environment and controlling malodors. Fragrances used in free-standing gel formulations are generally continuously delivered into the air, assisted by the vaporization of water and other co-solvents. Many of the prior art gel air fresheners have a tendency to become opaque or cloudy in appearance due to, for example, phase separation of the gel composition during use. In recent years, transparent gel air fresheners have been widely marketed and have gained popularity; the cloudy appearance of some gels, however, makes them undesirable for decorative consumer use.

Transparent gel compositions that avoid the disadvantages of an opaque of cloudy appearance still may exhibit other problems. For example, gel structures often can be destabilized by one or more freeze-thaw cycles, which may result in degradation of appearance, configuration, and/or functionality of the gel air freshener product. Moreover, traditional transparent gels typically exhibit noticeable signs of syneresis, which gives the gel a wet appearance. Generally, syneresis results in separation of the aqueous medium from the fragranced gel, due to gel contraction and/or inadequate gel strength. This phenomenon is especially detrimental to the aesthetic appeal of the product, as most transparent gels are packaged in clear, transparent containers.

Additionally, air bubbles generated during manufacturing of prior art gel compositions tend to accumulate at the surface of the gel when the gel is in a molten liquid state during temperature processing. As the gel sets at lower temperature, the bubbles migrate to the surface and cause an apparent roughness or foaming on the gel surface. In addition to detracting from the visual appeal of the gel, the foam generated during processing can cause manufacturing difficulties during mixing and transferring of the molten composition.

Another disadvantage of gel air fresheners is temperature instability. For example, several prior art gel formulations can exhibit a lack of stability when exposed to the extreme temperatures encountered during storage and shipping. Such lack of stability is generally exhibited by, for example, product weight loss, degradation of product appearance, loss of fragrance effectiveness, etc. Specifically, the poor freeze-thaw stability of some prior art gel air freshener formulations is demonstrated by an increase in syneresis and corresponding reduction in gel size and appearance when these formulations are subjected to one or more freeze-thaw cycles. Moreover, some of the transparent gel air freshener formulations of the prior art are elastic and soft, and therefore may be less stable and resistant to physical degradation during storage, shipping, and handling.

Formulations and methods of preparing gel air fresheners, including transparent gel air fresheners, are well known in the prior art. Prior art gels generally contain water, a gelling agent, a cross-linking agent, a co-solvent, and fragrance. For example, U.S. Pat. No. 5,750,498 (Soeda et al.) discloses a transparent gel air freshener composition comprising gelatin, a cross-linking agent, water, perfume, a solvent, and a surfactant. U.S. Pat. No. 5,679,334 (Semoff et al.) teaches a transparent gel formulation comprising water, a fragrance oil, a nonionic surfactant, a gelling agent, a cross-linking agent, and a co-solvent. In the prior art, the addition of a cross- linking agent to the gelling agent and other gel components is recognized as critical to achieve the desired gel properties, such as optimum clarity and gel structure.

In light of the disadvantages of the prior art, a composition is needed that will provide a substantially transparent, physically stable and aesthetically pleasing gel having a fragrance component which is dispensed over time into the surrounding environment without increasing the manufacturing cost or complexity.

### SUMMARY OF THE INVENTION

This invention generally relates to substantially transparent, substantially solid, free-standing gel compositions for dispensing functional volatile materials such as fragrance oils, fragrance solutions, deodorants, insecticides and the like into the surrounding air. While the way in which the present invention addresses the disadvantages of the prior art will be discussed in greater detail below, in general, the gel compositions of the present invention do not include added cross-linking and/or chelating agents. Surprisingly, exemplary compositions of the present invention have been found to demonstrate improved hardness, physical stability, and syneresis-resistancy.

In accordance with various exemplary embodiments of the present invention, chelating agents, such as, but not limited to, EDTA, EDHP, and citrate salts, are preferably not incorporated into the gel formulations either. In contrast to prior art teachings, surprisingly the present inventors herein demonstrate that the incorporation of chelating agents tends to have detrimental effects on gel strength and syneresis. In accordance with various aspects of the present invention, the hardness and resistance to syneresis of preferred translucent gel air freshener compositions is improved by employing a modified polysaccharide as the gelling agent without the addition of cross-linking agents.

The present invention, in accordance with various other embodiments, also discloses the use of an amine oxide as part of the gel composition. In accordance with various aspects of the embodiment, the present inventors have found that by controlling the amine oxide to nonionic co-surfactant ratio and the volatile material to total surfactant ratio, the overall surfactant level of the product gel composition can be reduced and gel stability, as well as other properties of the resulting gel, can be improved.

In accordance with still further exemplary embodiments of the present invention, the gel compositions preferably include a nonionic-based silicone antifoam agent to facilitate gel processing and improve surface smoothness of the final product. Preferred nonionic silicone antifoaming agents useful in the gel compositions of the present invention are preferably suitably selected and incorporated into the specific formulation such that gel strength, gel stability, and gel clarity are not negatively affected.

### DETAILED DESCRIPTION OF EXEMPLARY EMBODIMENTS

The following descriptions are of exemplary embodiments of the invention only, and are not intended to limit the scope, applicability or configuration of the invention in any way. Rather, the following description is intended to provide convenient illustrations for implementing various embodiments of the invention. As will become apparent, various changes may be made in the function and arrangement of the elements described in these embodiments without departing from the sprit and scope of the invention.

In general, transparent, free-standing gel compositions for dispensing functional volatile materials such as fragrance oils, fragrance solutions, deodorants, insecticides and the like, from the gel composition into the surrounding air are disclosed in accordance with the various embodiments of the present invention. In that regard, transparent gel compositions and the preparation method for achieving a gel air freshener having superior gel properties, such as hardness, physical stability, and syneresis-resistancy will herein be disclosed. In a broad sense, such compositions include any transparent gel air freshener composition that eliminates the use of additional cross-linking agents and/or chelating agents in combination with the gelling agent to improve gel strength and stability.

The strength of a gel composition may be thought of in terms of (1) the gel's ability to resist persistent deformation, in the case of a high modulus gel; or (2) the gel's ability to resist rupturing, in the case of a more flexible gel composition. Generally, the gel strength may be measured by determining the force (i.e., in grams per unit surface area) required to rupture or persistently deform the surface of the sample. Gel strength values, found in Tables 1-3, are given in units of grams of force for a rupture probe of constant surface area between the trial measurements.

Prior art techniques for forming stable gel compositions generally involve the addition of chelating agents to reduce the ion concentration down to a baseline level and later, cross-linking agents to elevate the ion concentration of the gel to a performance range that maximizes gel strength. In accordance with one exemplary aspect of the present invention, the use of chelating agents to purge ions from solution only to later re-populate ion concentration at a later point in the manufacturing process is not only not required, but also not preferred. The prior art, for example, in U.S. Pat. No. 5,679,334 (Semoff et al.), requires the presence of added cross-linking agents to enhance the ion concentration for manufacture of a gel composition. Additionally, the prior art suggests that the addition of ionic cross-linking agents to standard gel formulations enhance gel strength by maintaining cationic concentration and the concentration of monovalent cations such as Na+, K+, etc. According to such prior art teachings, altering the cationic concentration within an air freshener gel composition may affect the physical properties of the gel, for example, setting temperature, melting temperature, clarity, hardness, and brittleness. The concepts of “hardness” and/or “brittleness”, as they refer to properties of gel compositions herein described, are measures of rigidity such that a hard or brittle gel is generally considered to be sufficiently rigid so as to resist significant translational movement within a contained environment, yet flexible enough to permit vibrational resonance in accordance with non-destructive eigen-modes of the gel volume and geometry. Moreover, these prior art teachings also suggest that altering the concentration of monovalent cations within the gel composition may affect the ability of the gel composition to resist syneresis. Contrary to these prior art teachings, the present inventors have found that gel compositions containing a lower concentration of available ions exhibit an even higher level of gel strength when residual divalent ions are used to develop and build the gel matrix. Low ion concentration, in accordance with various aspects of the present invention preferably are less than about 50 parts per million (ppm), and, as will be discussed in greater detail below, such ion concentrations ranges preferably range up to about 50 parts per million (ppm), more preferably up to 35 ppm and most preferably up to about 25 ppm. The present inventors have discovered that controlling the ion concentration of the gel composition such that it generally remains within this range tends to maximize gel strength and minimize the raw material cost associated with the additional steps of (1) chelating out ions to establish a premix baseline ion concentration; and (2) adding ionic cross-linking agents to establish an effective ion concentration for cross-linking the gel matrix. In accordance with various aspects of the present invention, controlling the ion concentration of the gel composition may be achieved by allowing the divalent ions present in the gelling agent and other ingredients, as they are received from suppliers, to cross-link the gel matrix. Alternatively, efforts may be made to add and/or eliminate the presence of, and accordingly, the concentration of available monovalent cations.

For example, as will be described in greater detail herein, in accordance with one embodiment of the present invention, a gelling agent for use in connection with an air freshener composition comprises a low acyl gellan gum that forms firm gels at low concentrations. For example, gellan gums having a low acyl polysaccharide produced by the microorganism Sphingomonas elodea through fermentation and deacylation may be advantageously used. It has been found that such gelling agents exhibit surprisingly high gel strength and optical clarity. Preferred gelling agents useful in accordance with various aspects of this embodiment of the present invention include those sold under the trademarks KelcoGel® and KelcoGel® AFT, available from Kelco Biopolymers of San Diego, Calif.

In accordance with one preferred embodiment of the present invention, the gelling agent is present in the gel composition from about 0.3% to about 3.0% by weight of the total composition, preferably from about 0.6% to about 2.0% by weight. At levels below about 0.3 wt. %, the gel composition may not provide adequate hardness to support a free-standing gel and therefore difficulties in shipping and handling may arise. On the other hand, gelling agent concentrations above about 3.0 wt. % may unsuitably increase the raw material and processing costs of the gel composition. While in certain cases, gelling agent concentrations may exceed about 3.0 wt. %, costs may be increased without commensurate increases in product characteristics.

In general, air freshener compositions in accordance with the present invention include the gelling agent, a nonionic surfactant, and a fragrance oil. For example, in one embodiment of the present invention, the surfactant comprises a linear alkyl-ethoxylated nonionic surfactant with an BLB (hydrophilic-lipophilic balance) of between about 12 and about 18, preferably between about 14 and about 16, for example, Tomadol 25-12 supplied by Tomah. Preferably, the gel air freshener compositions of the present invention comprise from about 2% to about 20% by weight of surfactant, preferably from about 7% to about 15%. The nonionic surfactant is used to optimize the interference of the surfactant with the gelling gum. Preferably, the usage level of the surfactant is appropriately selected to ensure emulsification of the high level of fragrance oils and the antifoaming agent, as well as to maintain the clarity of the gel as will be described herein in connection with the various examples of preferred gel compositions.

In accordance with various aspects of the present invention, fragrance oils may comprise any material having different degrees of volatility. In one embodiment of the present invention, the gel air freshener composition comprises from about 1% to about 10% by weight of fragrance, preferably from about 3% to about 5%. Preferably, fragrance oils are selected such that they can be emulsified by the specific type and level of surfactants in the aqueous base so that the gel formula maintains its substantially transparent appearance.

It is preferable that the gel formulations of the present invention do not incorporate separately-added cross-linking agents or chelating agents. Addition of these cross-linking agents or chelating agents tend to reduce the gel hardness and/or resistance to syneresis, as shown by the Examples. In this context, cross-linking agents include, but are not limited to, citrate salts such as potassium citrate, and chloride salts such as sodium chloride, potassium chloride and magnesium chloride. Chelating agents include, but are not limited to, citrate salts such as potassium citrate, EDTA, and EDHP. In particular, citrate salts such as potassium citrate should be avoided because they significantly reduce the capability of the gel to resist syneresis.

The gel formulations of the invention preferably include a nonionic, silicone-based antifoaming agent. In the antifoaming agent, polydimethylsiloxane is emulsified by other nonionic surfactants and nonionic rheology modifiers as supplied. Such nonionic, silicone-based antifoaming agents can be found, for example, under the tradename Dow Corning Antifoam B Emulsion or 7305 Antifoam Emulsion supplied by Dow Corning Company. Preferably, the nonionic antifoaming agent is selected such that it does not interfere with the gelling gum. The nonionic based antifoaming system should also be compatible with the nonionic surfactant used in the clear gel formulation discussed previously. Preferred range of usage levels is from about 0.0001 to about 0.1%, and more preferably from about 0.005 to about 0.02%.

Minor but effective amounts of adjuvants such as color dyes, preservatives, bactericides, aversive agents, and the like may also be present in the gel air freshener formulations of the present invention. Other ingredients, such as preservatives and UV inhibitors may also be incorporated into the gel formulation. Incorporating a suitable UV inhibitor is preferable, as the transparent gel formulation is usually contained in a clear transparent container to enhance the decorative effect, and thus is exposed to UV light. This may result in discoloring of the product during use if UV inhibitors are not added. A preferred UV inhibitor is Benzophenone-2, which may be incorporated from about 0.005 to about 0.02%. This preferred UV inhibitor may be emulsified in the gel formulation without negatively affecting the other gel properties. A preferred preservative is Kathon CG or Kathon CG/CP, which may be incorporated from about 0.02 to about 0.2%.

In accordance with various additional aspects of the present invention, optional ingredients such as co-solvents and/or volatile alcohols may be added to the gel composition to enhance desirable gel properties such as optical clarity, and/or to enhance the fragrance delivery rate. Co-solvents may be used to assist in the movement of the fragrance components through the gel matrix, and thus tend to moderate the evaporation rate of the fragrance and assist in maintaining consistency in fragrance delivery to the surrounding environment. Preferably, the co-solvent is water-soluble. Preferred co-solvents include, for example, propylene glycol, dipropylene glycol, other glycol ethers, isopropylmyristate, diethyl phthalate, benzyl alcohol, benzyl benzoate, glyceryl triacetate, ethanol, isopropanol and the like. In addition to these preferred co-solvents, compounds such as diethyl phthalate, benzyl alcohol, benzyl benzoate, and glyceryl triacetate may be suitable as co-solvents to moderate the evaporation rate of the fragrance. Compounds such as ethanol and isopropanol may also be used as co-solvents to enhance the evaporation rate of the fragrance from the gel composition.

In a preferred exemplary embodiment of the present invention, a free-standing, substantially hard gel air freshener may be prepared by the following method. First, the gelling gum is dispersed in one or more co-solvents to form a slurry, which is then added to water, heated to approximately 185° F. and mixed until dissolved. While the gelling agent will readily disperse into solution, the kinetics of the reaction are such that the gelling agent must generally be mixed in an aqueous phase for a given amount of time (i.e., about 5 minutes) at an elevated temperature (i.e., about 180° F.) before the gel will begin to set. Next, the mixture is allowed to cool slightly to about 170-175° F., and the nonionic surfactant, antifoaming agent, and other optional ingredients (such as a preservative and/or a UV inhibitor) are added and mixed until dissolved. The mixture is allowed to cool slightly once more to about 145-170° F., then the fragrance is added and mixed until emulsified by the surfactant. The entire batch is cooled to about 140-145° F. and mixed until homogeneous. Finally, the cooled mixture is poured into the desired containers and cooled further to approximately 75-80° F. to allow the gel to further set. Residual divalent ions in the gelling gum and other ingredients are used to build and develop gel strength.

Table 1, set forth below, provides one exemplary formulation useful in accordance with the present invention (identified in Table 1 as Example 1). The Comparative Examples (2-4 in Table 1) are provided to illustrate various surprising and unexpected aspects of the present invention, which as will be understood, can be accomplished in a variety of ways.

TABLE 1

| | 1 | 2 | 3 | 4 |
|---|---|---|---|---|
| Ingredients | (Example) | (Comparative) | (Comparative) | (Comparative) |
| Formulation | | | | |
| KelcoGel AFT (%) | 1 | 1 | 1 | 1 |
| Propylene glycol (%) | 4 | 4 | 4 | 4 |
| Antifoam B emulsion | 0.01 | 0.01 | 0.01 | 0.01 |
| (%) | | | | |
| Nonionic surfactant | 13 | 13 | 13 | 13 |
| (Tomadol 25-12) (%) | | | | |
| Perfume - BBA 994624 | 5 | 5 | 5 | 5 |
| (%) | | | | |
| Color dyes (%) | 0.000169 | 0.000169 | 0.000169 | 0.000169 |
| Benzophenone-2 (%) | 0.01 | 0.01 | 0.01 | 0.01 |
| Kathon CG (%) | 0.1 | 0.1 | 0.1 | 0.1 |
| Potassium citrate (%) | 0 | 0.2 | 0.4 | 0.6 |
| Deionized water (%) | Add to 100 | Add to 100 | Add to 100 | Add to 100 |
| Results | | | | |
| Gel Strength (g-force) | 1000 | 403 | 658 | 824 |
| Gel Strength (after | 200 | 70 | N/A | 140 |
| freeze-thaw) | | | | |
| Syneresis weight loss | 0.01 | N/A | N/A | N/A |
| after 1 cycle of freeze- | | | | |
| thaw (% wt.) | | | | |
| Syneresis weight loss | 0.27 | N/A | N/A | N/A |
| after 2 cycles of freeze- | | | | |
| thaw (% wt.) | | | | |
| Syneresis weight loss | 0.35 | 3.37 to 19.40 | N/A | N/A |
| after 3 cycles of freeze- | | | | |
| thaw (% wt.) | | | | |

Table 1 demonstrates, among other things, that when the potassium citrate level is increased from 0 (1-Example) to 0.2% (2-Comparative), 0.4% (3-Comparative), and 0.6% (4-Comparative), the gel strength is lowered and syneresis is increased. This data illustrates that added cross-linking agent (e.g., potassium citrate) has a negative impact on the gel structural integrity, and thus is preferably not incorporated into the gel of the present invention.

Table 2, set forth below, provides a further exemplary formulation useful in accordance with the present invention (identified in Table 2 as Example 5). The Comparative Examples (6-7 in Table 2) are provided to illustrate various surprising and unexpected aspects of the present invention, which as will be understood, can be accomplished in a variety of ways.

TABLE 2

| | 5 | 6 | 7 |
|---|---|---|---|
| Ingredients | (Example) | (Comparative) | (Comparative) |
| Formulation | | | |
| KelcoGel AFT (%) | 1 | 1 | 1 |
| Propylene glycol (%) | 4 | 4 | 4 |
| Antifoam B emulsion | 0.01 | 0.01 | 0.01 |
| (%) | | | |
| Nonionic surfactant | 13 | 13 | 13 |
| (Tomadol 25-12) (%) | | | |
| Perfume - BBA 994621 | 5 | 5 | 5 |
| (%) | | | |
| Color dyes (%) | 0.000169 | 0.000169 | 0.000169 |
| Benzophenone-2 (%) | 0.01 | 0.01 | 0.01 |
| Kathon CG (%) | 0.1 | 0.1 | 0.1 |
| Potassium citrate (%) | 0 | 0.2 | 0.4 |
| Deionized water (%) | Add to 100 | Add to 100 | Add to 100 |
| Results | | | |
| Gel Strength (g-force) | 596 | 576 | Disintegrated |
| Gel Strength (after | 145 | 110 | Disintegrated |
| freeze-thaw) | | | |
| Syneresis weight loss | 0.01 | N/A | N/A |
| after 1 cycle of freeze- | | | |
| thaw (% wt.) | | | |
| Syneresis weight loss | 0.49 | N/A | N/A |
| after 2 cycles of freeze- | | | |
| thaw (% wt.) | | | |
| Syneresis weight loss | 0.27 | 3.37 to 19.40 | N/A |
| after 3 cycles of freeze- | | | |
| thaw (% wt.) | | | |

Table 2 demonstrates, among other things, that when potassium citrate is added to certain gel formulations that similar results as in Example 1 are found. Added citrate salt significantly reduces gel strength and increases syneresis, and thus is preferably not incorporated into the gel of the present invention.

A similar effect is seen when a different cross-linking agent, for example, potassium chloride, is added to the composition set forth as Formulation 5 (Table 2). This effect is shown

TABLE 3

| | | Gel Strength after freeze-thaw |
|---|---|---|
| Level of KCl | Gel Strength (g-force) | (g-force) |
| 0 | 596 | 145 |
| 0.05 | 525 | 122 |
| 0.10 | 535 | 135 |
| 0.15 | 272 | 82 |
| 0.20 | Disintegrated | Disintegrated |

In an exemplary preferred embodiment of the present invention, a first premix is prepared by adding 431.6 lbs. of Tomadol 25-12 to a premix tank equipped with a marine-type propeller mixer (e.g., lightning mixer). As the Tomadol 25-12 is slowly agitated, 166 lbs. of fragrance is added with continued mixing. Thereafter, 0.332 lbs. of benzophenone-2 is added with continued mixing for an additional 10 minutes. The Tomadol/fragrance/benzophenone premix is then heated to 110° F. to ensure that the mixture is substantially clear. A second premix is then prepared by adding 132.8 lbs. of propylene glycol to a second premix tank also equipped with a propeller mixer. As the propylene glycol is agitated, 33.2 lbs. of gellan gum is slowly introduced into the mixture with continued agitation until the mixture is substantially homogeneous. A third premix is then prepared by adding 0.00561 lbs. of dye to 10 lbs. of purified water in a stainless steel container with agitation until the mixture is substantially homogeneous. 2515.75 lbs. of purified water is added to a steam-jacketed, stainless steel main batch mix tank equipped with side sweeps and a central agitator. Thereafter, the propylene glycol/gellan gum premix is added and agitated for 5 minutes. The main batch is then heated to 185° F. with continued mixing and is further agitated for an additional 15 minutes. The main batch mixture appears clear when the gellan gum is substantially completely hydrated. The main batch is then cooled to 170° F. to prepare the mixture for addition of the fragrance/surfactant premix. The dye premix is then added with agitation until the color is substantially uniformly dispersed. After adding the dye premix to the main batch, the dye premix container is then rinsed with another 10 lbs. of purified water which is also added to the main batch. 0.332 lbs. of antifoam is then added to 16.66 lbs. of purified water with mixing until substantially homogeneous. The antifoam solution is then added to the main batch tank with continued mixing. After the main batch temperature has cooled to 170° F., the temperature is stabilized to prevent localized gelling during the addition of the Tomadol/fragrance/benzophenone premix with controlled heating of the main batch tank jacket. 3.32 lbs. of Kathon CG/ICP is then added with mixing. Thereafter, the Tomadol/fragrance/benzophenone premix is added slowly down the side sweeps of the main batch tank to avoid aerating the batch. The main batch is then mixed for an additional 10 minutes with care to maintain the tank jacket temperature above 160° F. The main batch is then passed through a 5 micron polyester bag filter and transferred to a preheated filler hold/head tank to avoid gelling of the product during the transfer. The batch is then cooled and temperature stabilized to 145±5° F. during filling with adequate mixing in the hold/head tank to avoid separation. Generally, the fill temperature should remain between 135° F. and 140° F.

The process equipment and pipelines in contact with the finished product are preferably fabricated of Type #304 stainless steel. Additionally, the equipment and pipelines should be in a clean condition and substantially free of water, gel (finished product), and other liquids or powders.

In addition to the foregoing compositions which embody various aspects of the present invention, it has been discovered that different lots of gelling agent often exhibit different properties, for example, with respect to gel strength. It is believed that such differences relate to the presence, or absence, as the case may be, of naturally-occurring monovalent cations that may be present in the gelling agent. For example, different lots of KelcoGel were used to prepare the air freshener composition identified as Formulation 5 (Table 2) to examine the effect of the addition of potassium chloride. As shown in Table 4, set forth below, the data illustrates that potassium chloride tends to reduce the gel strength independent of the variations in the gelling gum.

TABLE 4

| Gelling Gum | | |
|---|---|---|
| Lot No. | Potassium Chloride Concentration | Gel Strength (g-force) |
| Lot A | No KCl separately added | 855 |
| Lot A | 0.5% KCl | 244 |
| Lot B | No KCl separately added | 900 |
| Lot B | 0.5% KCl | 231 |

As depicted in Table 5, five exemplary gel formulations A-E were prepared containing the amine oxide Standamox CAW (cocamidopropylamine oxide) and a nonionic surfactant Tomadol 25-12.

TABLE 5

| | A | B | C | D | E |
|---|---|---|---|---|---|
| Material: | (% wt.) | (% wt.) | (% wt.) | (% wt.) | (% wt.) |
| Gellan Gum | 1.000 | 1.000 | 1.000 | 1.000 | 1.000 |
| Propylene glycol | 4.000 | 4.000 | 4.000 | 4.000 | 4.000 |
| Antifoam | 0.010 | 0.010 | 0.010 | 0.010 | 0.010 |
| UV inhibitor | 0.010 | 0.010 | 0.010 | 0.010 | 0.010 |
| Ethanol | 1.000 | 1.000 | 1.000 | 1.000 | 1.000 |
| Tomadol 25-12 | 2.600 | 4.600 | 2.600 | 3.600 | 2.250 |
| Standamox CAW (30% | 14.670 | 14.670 | 21.330 | 18.000 | 12.500 |
| active) | | | | | |
| Fragrance | 4.000 | 4.000 | 4.000 | 4.000 | 4.000 |
| Preservative | 0.100 | 0.100 | 0.100 | 0.100 | 0.100 |
| Dyes | 0.001 | 0.001 | 0.001 | 0.001 | 0.001 |
| Water | Add up | Add up | Add up | Add up | Add up |
| | to 100% | to 100% | to 100% | to 100% | to 100% |

By introducing an amine oxide surfactant and restricting the weight ratio with at least one co-surfactant, the total surfactant level of the gel composition may be reduced without substantially compromising gel stability, optical clarity or temperature stability. In various embodiments of the present invention, lower overall surfactant levels have been shown to be desirable because the surfactant matrices in the gel tend to slow down the delivery rate of functional volatile components and leave a chunky residue at the end of the product's life.

The exemplary gel formulations A-E, shown in Table 5, contain cocamidopropylamine oxide as the dominant surfactant for dissolving fragrances and other insoluble components. Cocamidopropylamine oxide is provided by Cognius under the tradename Standamox CAW. The weight ration between cocamidopropylamine oxide and the ethoxylated nonionic surfactant Tomadol 25-12, in one exemplary embodiment, is between about 1:2 and about 3:1 by weight. In an alternative exemplary embodiment, the amine oxide to nonionic co-surfactant ratio is between about 1:1 and about 3:1 by weight. In yet another preferred exemplary embodiment, the amine oxide to nonionic co-surfactant ratio is between 1.6:1 and 2.5:1 by weight.

Control of the total surfactant to fragrance weight ratio has also been demonstrated in exemplary embodiments of the present invention to help preserve gel stability at low overall surfactant levels. In one exemplary embodiment, the fragrance to total surfactant ratio is between about 1:1 and about 3:1 by weight. In a preferred alternative exemplary embodiment, the fragrance to total surfactant ratio is between about 1.5:1 and about 2:1.

Various principles and applications of the present invention have been described by way of the preceding exemplary embodiments; however, other combinations and/or modifications of the above-described structures, arrangements, applications, proportions, elements, materials or components used in the practice of the present invention, in addition to those not specifically recited, may be varied or otherwise particularly adapted by those skilled in the art to specific environments, manufacturing or design parameters or other operating requirements without departing from the general principles of the same. For example, while the foregoing is described using cocamidopropylamine as the amine oxide, alternative amine oxides such as alkyldimethylamine oxide, dimethyllaurylamine oxide, dimethylmyristylamine oxide, stearyldimethylamine oxide may likewise be used and still fall within the scope of the present invention.

We claim:

1. A gel composition, comprising:
about 0.3% to about 3.0% by weight of a modified polysaccharide gelling agent;
about 1.0% to about 10.0% by weight of a functional volatile component;
up to about 0.1% by weight of a nonionic based antifoam agent; and
about 2.0% to about 20.0% by weight of a nonionic surfactant,
wherein said composition is substantially free of cross-linking and chelating agents.

2. The gel composition of claim 1, wherein the modified polysaccharide gelling agent is a low acyl polysaccharide.

3. The gel composition of claim 2, wherein the modified polysaccharide gelling agent is a gellan gum.

4. The gel composition of claim 1, wherein the modified polysaccharide gelling agent is present in an amount of about 0.6% to about 2.0% by weight.

5. The gel composition of claim 1, wherein the functional volatile component is present in an amount of about 3% to about 5% by weight.

6. The gel composition of claim 1, wherein the functional volatile component is a fragrance.

7. The gel composition of claim 1, wherein the functional volatile component is an insecticide.

8. The gel composition of claim 1, wherein the antifoam agent is present in an amount of about 0.02% by weight.

9. The gel composition of claim 1, wherein the surfactant is a linear alkyl-ethoxylated nonionic surfactant having a hydrophilic-lipophilic balance of between about 12 and 18.

10. The gel composition of claim 9, wherein the surfactant is a linear alkyl-ethoxylated nonionic surfactant having a hydrophilic-lipophilic balance of between about 14 and 16.

11. The gel composition of claim 1, wherein the nonionic surfactant is present in an amount of about 7.0% to about 15.0% by weight.

12. The gel composition of claim 1, further comprising components selected from the group consisting of dyes, preservatives, bactericides, aversive agents, UV inhibitors, and any mixtures thereof.

13. The gel composition of claim 1, further comprising about 0.02% by weight of benzophenone-2.

14. The gel composition of claim 1, further comprising about 0.02% to about 0.2% of a preservative.

15. The gel composition of claim 1, further comprising a co-solvent.

16. The gel composition of claim 15, wherein the co-solvent is miscible with water.

17. The gel composition of claim 16, wherein the co-solvent is selected from the group consisting of propylene glycol, dipropylene glycol, diethyl phthalate, benzyl alcohol, benzyl benzoate, glyceryl triacetate, methyl propanediol, diethylene glycol monoethyl ether, isopropylmyristate, ethanol and isopropanol.

18. A gel air freshener composition, comprising:
about 0.3% to about 3.0% by weight of a modified polysaccharide gelling agent;
about 1.0% to about 10.0% by weight of a fragrance;
up to about 0.1% by weight of a nonionic based antifoam agent;
about 2.0% to about 20.0% by weight of a nonionic surfactant; and
an effective amount of an amine oxide wherein the weight ratio of amine oxide to nonionic surfactant is from about 1:2 to 3:1.

19. The gel composition of claim 18, wherein the weight ratio of amine oxide to nonionic surfactant is from about 1:1 to 3:1.

20. The gel composition of claim 19, wherein the weight ratio of amine oxide to nonionic surfactant is from about 1.5:1 to 2:1.

21. The gel composition of any one of claims 18, wherein the amine oxide is selected from the group consisting of cocamidopropylamine oxide, alkyldimethylamine oxide, dimethyllaurylamine oxide, dimethylmyristylamine oxide and stearyldimethylamine oxide.

22. The gel composition of any one of claims 18, wherein the surfactant is a linear alkyl-ethoxylated nonionic surfactant.

23. A substantially stable gel composition having a reduced total surfactant level, comprising an amine oxide and a nonionic co-surfactant having an amine oxide to co-surfactant weight ratio of from about 1:2 to 3:1.

24. The gel composition of claim 23, wherein the weight ratio of amine oxide to nonionic co-surfactant is from about 1:1 to 3:1.

25. The gel composition of claim 24, wherein the weight ratio of amine oxide to nonionic co-surfactant is from about 1.5:1 to 2:1.

26. A substantially stable, fragranced gel composition having a relatively low surfactant level, wherein the surfactant to fragrance weight ratio is from about 1:1 to 3:1.

27. The gel composition of claim 26, wherein the weight ratio of surfactant to fragrance is from about 1.5:1 to 2:1.

28. A method for manufacturing a gel composition comprising the steps of:
providing about 0.3% to about 3.0% by weight of a modified polysaccharide gelling agent;
providing about 1.0% to about 10.0% by weight of a functional volatile component;
providing up to about 0.1% by weight of a nonionic based antifoam agent; and
providing about 2.0% to about 20.0% by weight of a nonionic surfactant substantially free of cross-linking and chelating agents.

29. The method of claim 28, wherein the modified polysaccharide gelling agent is a low acyl polysaccharide.

30. The method of claim 29, wherein the modified polysaccharide gelling agent is a gellan gum.

31. The method of claim 28, wherein the modified polysaccharide gelling agent is present in an amount of about 0.6% to about 2.0% by weight.

32. The method of claim 28, wherein the functional volatile component is present in an amount of about 3% to 5% by weight.

33. The method of claim 28, wherein the functional volatile component is a fragrance.

34. The method of claim 28, wherein the functional volatile component is an insecticide.

35. The method of claim 28, wherein the antifoam agent is present in an amount of about 0.02% by weight.

36. The method of claim 28, wherein the surfactant is a linear alkyl-ethoxylated nonionic surfactant having a hydrophilic-lipophilic balance of between about 12 and 18.

37. The method of claim 28, wherein the surfactant is a linear alkyl-ethoxylated nonionic surfactant having a hydrophilic-lipophilic balance of between about 14 and 16.

38. The method of claim 28, wherein the nonionic surfactant is present in an amount of about 7.0% to about 15.0% by weight.

39. The method of claim 28, further comprising the step of providing components selected from the group consisting of dyes, preservatives, bactericides, aversive agents, UV inhibitors, and any mixtures thereof.

40. The method of claim 28, further comprising the step of providing about 0.02% by weight of benzophenone-2.

41. The method of claim 28, further comprising the step of providing about 0.02% to about 0.2% of a preservative.

42. The method of claim 28, further comprising the step of providing a co-solvent.

43. The method of claim 42, wherein the co-solvent is miscible with water.

44. The method of claim 43, wherein the co-solvent is selected from the group consisting of propylene glycol, dipropylene glycol, diethyl phthalate, benzyl alcohol, benzyl benzoate, glyceryl triacetate, methyl propanediol, diethylene glycol monoethyl ether, isopropylmyristate, ethanol and isopropanol.

45. A method for manufacturing a gel composition, comprising the steps of:
providing about 0.3% to about 3.0% by weight of a modified polysaccharide gelling agent;
providing about 1.0% to about 10.0% by weight of a fragrance;
providing up to about 0.1% by weight of a nonionic based antifoam agent;
providing about 2.0% to about 20.0% by weight of a nonionic surfactant; and
providing an effective amount of an amine oxide wherein the weight ratio of amine oxide to nonionic surfactant is from about 1:2 to 3:1.

46. The method of claim 45, wherein the weight ratio of amine oxide to nonionic surfactant is from about 1:1 to 3:1.

47. The method of claim 46, wherein the weight ratio of amine oxide to nonionic surfactant is from about 1.5:1 to 2:1.

48. The method of any one of claims 45, wherein the amine oxide is cocamidopropylamine oxide.

49. The method of any one of claims 45, wherein the surfactant is a linear alkyl-ethoxylated nonionic surfactant.

50. A method for reducing the total surfactant level in a gel composition while substantially preserving gel stability, comprising the step of providing an amine oxide to nonionic co-surfactant weight ratio of from about 1:2 to 3:1.

51. The method of claim 50, wherein the weight ratio of amine oxide to nonionic co-surfactant is from about 1:1 to 3:1.

52. The method of claim 51, wherein the weight ratio of amine oxide to nonionic co-surfactant is from about 1.5:1 to 2:1.

53. A method for substantially preserving gel stability in a gel composition having a relatively low surfactant level, comprising the step of providing a surfactant to fragrance weight ratio of from about 1:1 to 3:1.

54. The method of claim 53, wherein the weight ratio of surfactant to fragrance is from about 1.5:1 to 2:1.

55. A method for providing a sustained release of at least one volatile air freshening component, comprising the steps of:
providing an air freshening article of manufacture having (a) about 0.3% to about 3.0% by weight of a modified polysaccharide gelling agent, (b) about 1.0% to about 10.0% by weight of at least one volatile air freshening component, (c) up to about 0.1% by weight of a nonionic based antifoam agent, and (d) about 2.0% to about 20.0% by weight of a nonionic surfactant; and
evaporating at least one volatile fragrance component from said air freshening article over time.

56. A method for providing a sustained release of at least one volatile air freshening component, comprising the steps of:
providing an air freshening article of manufacture having (a) about 0.3% to about 3.0% by weight of a modified polysaccharide gelling agent, (b) about 1.0% to about 10.0% by weight of at least one volatile air freshening component, (c) up to about 0.1% by weight of a nonionic based antifoam agent, (d) about 2.0% to about 20.0% by weight of a nonionic surfactant, and (e) an effective amount of an amine oxide wherein the weight ratio of amine oxide to nonionic surfactant is from about 1:2 to 3:1; and
evaporating at least one volatile fragrance component from said air freshening article over time.
